(19) United States
(12) Patent Application Publication
(10) Pub. No.: US 2022/0288331 A1
(43) Pub. Date: Sep. 15, 2022

(54) PHYSIOLOGICAL INFORMATION PROCESSING SYSTEM, LIGHT EMITTING APPARATUS AND PHYSIOLOGICAL INFORMATION PROCESSING APPARATUS

(71) Applicant: NIHON KOHDEN CORPORATION, Tokyo (JP)

(72) Inventors: Fumihiko TAKATORI, Tokorozawa-shi, Saitama (JP); Kentaro SUZUKI, Tokorozawa-shi, Saitama (JP); Masayuki INOUE, Tokorozawa-shi, Saitama (JP)

(21) Appl. No.: 17/754,154

(22) PCT Filed: Sep. 30, 2020
(86) PCT No.: PCT/JP2020/037343

§ 371 Date: Mar. 25, 2022

(30) Foreign Application Priority Data
Oct. 11, 2019 (JP) 2019-187882

(51) Int. Cl. A61M 16/00

(52) U.S. Cl. CPC A61M 16/0003; A61M 2205/584; A61M 2240/00; A61M 2230/42; A61M 2016/0027

(57) ABSTRACT

A physiological information processing system includes: a respiration sensor that is configured to detect a respiratory condition of a subject to whom a ventilation device is attached; a light emitting device that is configured to emit light toward an outside; and a physiological information processing apparatus that is connected to the respiration sensor and the light emitting device. The physiological information processing apparatus is configured to: acquire a parameter relevant to respiration of the subject; determine whether or not the parameter is included in a threshold range; and change a visual aspect of the light emitting device in response to determining whether or not the parameter is included in the threshold range. The light emitting device is removably attached to a columnar body.

### TECHNICAL FIELD

The present disclosure relates to a physiological information processing system, a light emitting device and a physiological information processing apparatus.

### BACKGROUND ART

In order to resuscitate a newborn baby who does not respire spontaneously or who is low in heart rate, non-invasive positive pressure ventilation using a bag mask may be carried out on the newborn baby at a medical site. For example, a non-invasive positive pressure ventilation apparatus which is constituted by a face mask and a respiration bag has been disclosed in WO 2009/047763. The face mask serves for covering the mouth and the nose of a patient such as a newborn baby. The respiration bag is connected to the face mask.

When a medical worker such as a doctor performs positive pressure ventilation on a patient by use of the positive pressure ventilation apparatus, it is difficult for the doctor to intuitively grasp whether the positive pressure ventilation is being appropriately performed on the patient or not. Particularly, when the positive pressure ventilation is performed on the patient who suffers cardiopulmonary arrest, it is possible to anticipate such a situation that the medical worker may hurry too much to appropriately perform the positive pressure ventilation on the patient. That is, it is possible to anticipate such a situation that the medical worker not noticing an abnormal situation which has occurred in the respiratory condition of the patient may continue the inappropriate positive pressure ventilation on the patient. Thus, there is still room for consideration about such a medical appliance through which the medical worker who is in the middle of operating a ventilation device can intuitively grasp the abnormality of the respiratory condition of the patient.

### SUMMARY

The present disclosure is directed to providing a physiological information processing system and a physiological information processing apparatus, through which a medical worker who is in the middle of operating a ventilation device can intuitively grasp abnormality of a respiratory condition of a subject. Further, the present disclosure is directed to providing a light emitting device, through which the medical worker who is in the middle of operating the ventilation device can intuitively grasp the abnormality of the respiratory condition of the subject or an operation timing of the ventilation device.

According to one or more aspects of the present disclosure, there is provided a physiological information processing system.

The physiological information processing system comprises:

a respiration sensor that is configured to detect a respiratory condition of a subject to whom a ventilation device is attached;

a light emitting device that is configured to emit light toward an outside; and

a physiological information processing apparatus that is connected to the respiration sensor and the light emitting device.

The physiological information processing apparatus is configured to:

acquire a parameter relevant to respiration of the subject;

determine whether or not the parameter is included in a threshold range; and

change a visual aspect of the light emitting device in response to determining whether or not the parameter is included in the threshold range.

The light emitting device is removably attached to a columnar body.

According to one or more aspects of the present disclosure, there is provided a light emitting device that is removably attached to a columnar body.

The light emitting device comprises:

a light emitting element that is configured to emit light;

a housing portion that houses the light emitting element and that allows at least a portion of the light emitted from the light emitting element to pass through the housing portion; and

an attachment portion that is connected to the housing portion and that is attached to the columnar body.

The light emitting device is configured to provide information indicating abnormality of a respiratory condition of the subject or operation guide information for guiding an operation timing of the ventilation device.

According to one or more aspects of the present disclosure, there is provided a physiological information processing apparatus that is connected to a light emitting device and a respiration sensor. The light emitting device is configured to emit light toward an outside. The respiration sensor is configured to detect a respiratory condition of a subject to whom a ventilation device is attached.

The physiological information processing apparatus is configured to:

acquire a parameter relevant to respiration of the subject;

determine whether or not the parameter is included in a threshold range; and

change a visual aspect of the light emitting device in response to determining whether or not the parameter is included in the threshold range.

The light emitting device is removably attached to a columnar body.

According to one or more aspects of the present disclosure, there is provided a physiological information processing system.

The physiological information processing system comprises:

a light emitting device that is configured to emit light toward an outside; and

a physiological information processing apparatus that is connected to the light emitting device.

The physiological information processing apparatus is configured to:

acquire a parameter relevant to respiration of a subject,

determine whether or not the parameter is included in a threshold range; and

change a visual aspect of the light emitting device in response to determining whether or not the parameter is included in the threshold range.

The light emitting device is removably attached to a columnar body.

According to the present disclosure, it is possible to provide a physiological information processing system and a physiological information processing apparatus, through which a medical worker who is in the middle of operating a ventilation device can intuitively grasp abnormality of a respiratory condition of a subject. Further, according to the present disclosure, it is possible to provide a light emitting device, through which the medical worker who is in the middle of operating the ventilation device can intuitively grasp the abnormality of the respiratory condition of the subject or an operation timing of the ventilation device.

### BRIEF DESCRIPTION OF DRAWINGS

FIG. 1 is a block diagram illustrating a physiological information processing system according to an embodiment of the present invention (which will be hereinafter referred to as “present embodiment” simply).

FIG. 2 is a schematic view illustrating a ventilation device and a respiration sensor attached to the ventilation device.

FIG. 3 is a flow chart for describing a process of changing a visual aspect of a light emitting device.

FIG. 4 is a perspective view illustrating the light emitting device and a measurement tube of the respiration sensor.

FIG. 5 is a top view illustrating the light emitting device and the measurement tube.

FIG. 6 is a perspective view illustrating the light emitting device attached to the measurement tube.

FIG. 7 is a schematic view illustrating a light emitting device according to a modification.

### DESCRIPTION OF EMBODIMENT

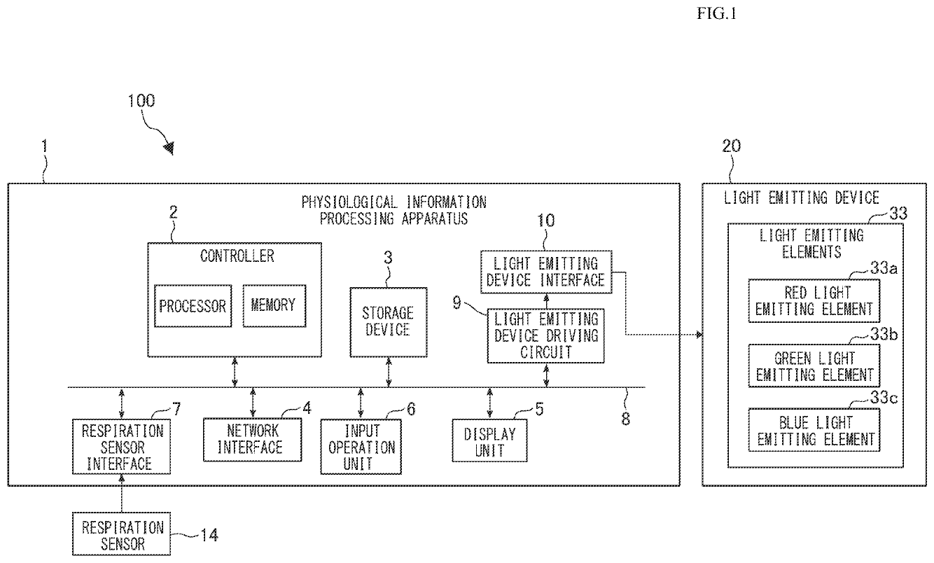

The present embodiment will be described below with reference to the drawings. In the first place, a physiological information processing system 100 according to the present embodiment will be described below with reference to FIG. 1. FIG. 1 is a block diagram illustrating the physiological information processing system 100 according to the present embodiment. As shown in FIG. 1, the physiological information processing system 100 comprises a physiological information processing apparatus 1 (which will be hereinafter referred to as processing apparatus 1 simply), a respiration sensor 14 which is connected to the processing apparatus 1, and a light emitting device 20 which is connected to the processing apparatus 1.

The processing apparatus 1 may be a physiological information monitor (bedside monitor) which is configured to display physiological information of a patient (e.g. information about a respiratory condition of the patient). In addition, the processing apparatus 1 may be a personal computer, a work station, a smartphone, a tablet, or a wearable device (such as AR glasses etc.) worn on the body (such as an arm or a portion of the head) of a medical worker.

The processing apparatus 1 is provided with a controller 2, a storage device 3, a network interface 4, a display unit 5, and an input operation unit 6. The processing apparatus 1 is further provided with a respiration sensor interface 7, a light emitting device driving circuit 9, and a light emitting device interface 10. The respective constituent elements of the processing apparatus 1 may be communicably connected to one another through a bus 8.

The controller 2 is provided with a memory and a processor. The memory is configured to store a computer-readable command (program). For example, the memory may be constituted by an ROM (Read Only Memory) in which various programs etc. have been restored, and an RAM (Random Access Memory) or the like which has a plurality of work areas in which the various programs etc. executed by the processor can be stored. In addition, the memory may be constituted by a flash memory etc. The processor includes, for example, at least one of a CPU (Central Processing Unit), an MPU (Micro Processing Unit), and a GPU (Graphics Processing Unit). In addition, the processor may include an FPGA (Field-Programmable Gate Array) and/or an ASIC (Application Specific Integrated Circuit). The CPU may be constituted by a plurality of CPU cores. The GPU may be constituted by a plurality of GPU cores. The processor may be configured to expand, onto the RAM, a physiological information program designated from the various programs which have been incorporated into the storage device 3 or the ROM, and to execute various processes in cooperation with the RAM.

The storage device 3 is, for example, a storage device (storage) such as an HDD (Hard Disk Drive), an SSD (Solid State Drive), or a flash memory, which is configured to store the programs or various data. The physiological information processing program may be incorporated into the storage device 3.

The network interface 4 is configured to connect the processing apparatus 1 to a communication network. Specifically, the network interface 4 may include various wired connection terminals for making communication with an external server through the communication network (such as an LAN, a WAN or the Internet). In addition, the network interface 4 may include a communication processing circuit such as an RF circuit, a transmission/reception antenna, etc. for making wireless communication with an access point (such as a wireless LAN router or a wireless base station). A wireless communication standard between the access point and the processing apparatus 1 is, for example, Wi-Fi (registered trademark), Bluetooth (registered trademark), Zigbee (registered trademark), LPWA or a fifth-generation mobile communication system (5G).

The display unit 5 may be a display device such as a liquid crystal display or an organic EL display. In addition, the display unit 5 may be a display device such as a transmission type or non-transmission type head mount display or an AR display which can be worn on the head of an operator. Further, the display unit 5 may be a projector device projecting an image on a screen. In addition, the processing apparatus 1 may be not provided with the display unit 5. In this case, an external display device may display image data transmitted from the processing apparatus 1 after the processing apparatus 1 transmits the image data to the external display device by wire or by wireless.

The input operation unit 6 is configured to accept an input operation of the medical worker operating the processing apparatus 1 and to generate an instruction signal in accordance with the input operation. The input operation unit 6 is, for example, a touch panel disposed to be superimposed on the display unit 5, a mouse, a keyboard, and/or a physical operation button etc. disposed on a housing of the processing apparatus 1. After the instruction signal generated by the input operation unit 6 is transmitted to the controller 2 through the bus 8, the controller 2 executes a predetermined operation in accordance with the instruction signal.

The respiration sensor interface 7 is an interface for connecting the respiration sensor 14 and the processing apparatus 1 to each other. The respiration sensor interface 7 may be physically connected to a connector of the respiration sensor 14. In addition, the respiration sensor interface 7 may have an analog processing circuit including an amplifier and an A/D converter, and a digital processing circuit including a processor such as a CPU and a memory such as an ROM. The respiration sensor interface 7 is configured to generate respiration data (digital data) based on an output result representing a respiratory condition of the patient acquired by the respiration sensor 14.

The respiration data may include airway pressure data representing temporal change of airway pressure and ventilation volume data representing temporal change of a ventilation volume. In addition, the respiration data may be expired gas concentration data etc. Details of the respiration sensor 14 will be given later.

The light emitting device driving circuit 9 is configured as a driving circuit for controlling driving of the light emitting device 20. The light emitting device driving circuit 9 is configured to transmit a lighting signal to the light emitting device 20 through the light emitting device interface 10 after the lighting signal supplied to the light emitting device 20 is generated in accordance with a control signal transmitted from the controller 2. The lighting signal supplied by the light emitting device driving circuit 9 is, for example, a pulse width modulation (PWM) signal.

The light emitting device 20 is configured to emit light toward the outside. The light emitting device 20 has a plurality of light emitting elements 33. The light emitting elements 33 are configured by a red light emitting element 33a (e.g. a red LED) emitting red light, a green light emitting element 33b (e.g. a green LED) emitting green light, and a blue light emitting element 33c (e.g. a blue LED) emitting blue light.

For example, assume a case where the light emitting device 20 emits white light. In this case, the light emitting device driving circuit 9 transmits the lighting signal to each of the red light emitting element 33a (e.g. the red LED), the green light emitting element 33b (e.g. the green LED) and the blue light emitting element 33c (e.g. the blue LED) which are provided in the light emitting device 20. Since the light emitting device 20 emits the red light, the green light and the blue light respectively in this manner, the white light is emitted toward the outside from the light emitting device 20.

The light emitting device interface 10 is an interface for connecting the light emitting device 20 and the processing apparatus 1 to each other. The light emitting device interface 10 may be physically connected to a connector of an electric cable attached to the light emitting device 20.

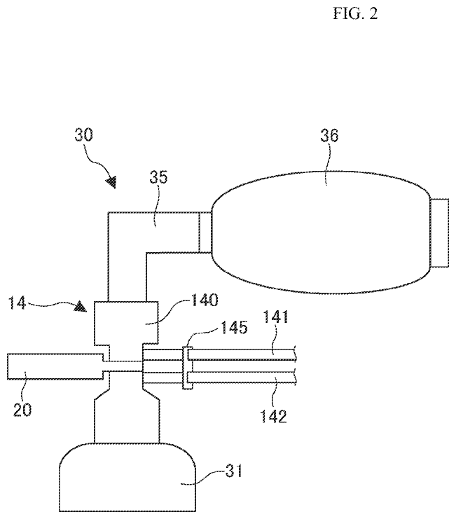

Next, an example of the structure of a ventilation device 30 and the respiration sensor 14 will be described below with reference to FIG. 2. FIG. 2 is a schematic view illustrating the ventilation device 30 and the respiration sensor 14 attached to the ventilation device 30. As shown in FIG. 2, the ventilation device 30 has a face mask 31, an air duct 35, and a respiration bag 36. The ventilation device 30 is a ventilation medical appliance for non-invasively performing positive pressure ventilation on a patient such as a newborn baby who does not respire spontaneously or who is low in heart rate. The medical worker (user) can perform positive pressure ventilation on the patient manually or automatically (semi-automatically) by use of the ventilation device 30.

The face mask 31 is configured to cover the mouth and the nose of the patient (such as the newborn baby). The air duct 35 is configured to allow air supplied from the respiration bag 36 to pass through. A backflow valve or a bacterial filter may be provided in the air duct 35. The respiration bag 36 communicating with the air duct 35 is configured to supply air to the patient in accordance with an operation of the medical worker. In this respect, the medical worker squeezes the respiration bag 36 with his/her hands so that air is supplied to the patient through the air duct 35, a measurement tube 140 and the face mask 31.

Next, the respiration sensor 14 will be described. The respiration sensor 14 is configured to detect the respiratory condition of the patient (subject) to whom the ventilation device 30 is attached. Particularly, the respiration sensor 14 is configured to detect airway pressure and a ventilation volume of the patient.

The respiration sensor 14 may be, for example, a flow sensor. As an example of the configuration of the respiration sensor 14, the respiration sensor 14 has the measurement tube 140 (an example of a columnar body) connected to the face mask 31 and the air duct 35, an inspiration side air tube 141, an expiration side air tube 142, and a connection portion 145 connecting the inspiration side air lube 141 and the expiration side air tube 142 with the measurement pipe 140.

The measurement tube 140 which is configured as a circularly columnar tube and communicates with the air duct 35 and the face mask 31. A variable orifice moving according to an inspiration side (respiration bag 36 side) flow or an expiration side (face mask 31 side) flow may be provided inside the measurement tube 140.

The inspiration side air tube 141 is configured to transmit flow and pressure of inspiration side air of the measurement tube 140. The expiration side air tube 142 is configured to transmit flow and pressure of expiration side air of the measurement tube 140. In addition, a differential pressure sensor is provided in one of the respiration sensor 14 and the respiration sensor interface 7. The differential pressure sensor is configured to detect differential pressure between the pressure of the inspiration side air and the pressure of the expiration side air.

The analog processing circuit of the respiration sensor interface 7 may convert, into digital data, differential pressure data representing the differential pressure between the pressure of the inspiration side air and the pressure of the expiration side air, which has been acquired by the differential pressure sensor. Then, the digital processing circuit of the respiration sensor interface 7 may acquire respiration data (airway pressure data and ventilation volume data) based on the differential pressure data (the digital data).

In addition, in the present embodiment, the light emitting device 20 is removably attached to the measurement tube 140 which is formed as a columnar body, as shown in FIG. 2. The specific structure of the light emitting device 20 will be described later.

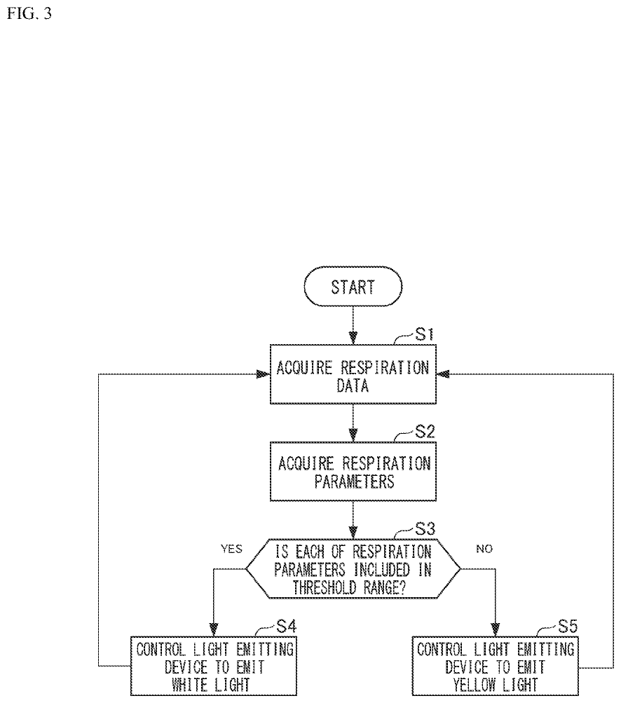

Next, a process of changing a visual aspect of the light emitting device 20 will be described below with reference to FIG. 3. FIG. 3 is a flow chart for describing the process of changing the visual aspect of the light emitting device 20. In a step S1, as shown in FIG. 3, the controller 2 (see FIG. 2) of the processing apparatus 1 acquires respiration data (e.g. airway pressure data and ventilation volume data) of a patient on whom the ventilation device 30 has been mounted, from the respiration sensor 14 and the respiration sensor interface 7. Here, the respiration data are digital data which have been AD-converted.

Next, in a step S2, the controller 2 acquires a plurality of respiration parameters relevant to respiration of the patient from the acquired respiration data. In this respect, the controller 2 acquires parameters relevant to the ventilation volume of the patient, parameters relevant to the airway pressure of the patient, and a parameter relevant to a respiration rate of the patient, as the respiration parameters.

(Parameters Relevant to Ventilation Volume of Patient)

The controller 2 acquires the parameters relevant to the ventilation volume of the patient based on the ventilation volume data which are an example of the respiration data. Particularly, the controller 2 acquires an inspired tidal volume (VTi) sent from the ventilation device 30 to the patient and an expired tidal volume (VTe) returned from the patient to the ventilation device 30. Further, the controller 2 calculates a leak ratio (%) representing a ratio of air leaking out from the face mask 31 based on the VTi and the VTe. Here, the leak ratio is calculated as (VTi−VTe)/VTi×100%.

(Parameters Relevant to Airway Pressure of Patient)

In addition, the controller 2 identifies the parameters relevant to the airway pressure of the patient based on the airway pressure data which are an example of the respiration data. Particularly, the controller 2 identifies peak inspiratory pressure (PIP) which is highest airway pressure in a respiration cycle, and positive end-expiratory pressure (PEEP).

(Parameter Relevant to Respiration Rate of Patient)

In addition, the controller 2 identifies the parameter relevant to the respiration rate of the patient based on the airway pressure data. That is, the controller 2 identifies the respiration rate (RR) of the patient.

Next, in a step S3, the controller 2 determines whether each of the respiration parameters is included or not in a threshold range. In this respect, the controller 2 determines whether each (e.g. the VTe or the leak ratio) of the parameters relevant to the ventilation volume of the patient is included or not in a threshold range. For example, assume that the threshold range of the VTe is set to be not lower than 0 mL and not higher than 20 mL. In this case, the controller 2 determines whether the acquired VTe is included or not in the threshold range which is not lower than 0 mL and not higher than 20 mL.

In addition, the controller 2 determines whether each of the parameters (e.g. PIP and PEEP) relevant to the airway pressure of the patient is included or not in a threshold range. For example, assume that the threshold range of the PIP is set to be not lower than 8 mmHg and not higher than 40 mmHg. In this case, the controller 2 determines whether the acquired PIP is included or not in the threshold range which is not lower than 8 mmHg and not higher than 40 mmHg. Further, assume that the threshold range of the PEEP is set to be not lower than 0 mmHg and not higher than 25 mmHg. In this case, the controller 2 determines whether the acquired PEEP is included or not in the threshold range which is not lower than 0 mmHg and not higher than 25 mmHg.

Further, the controller 2 determines whether the parameter (RR) relevant to the respiration rate of the patient is included or not in a threshold range. For example, assume that the threshold range of the RR is set to be not lower than 40 times/minute and not higher than 60 times/minute. In this case, the controller 2 determines whether the identified RR is included or not in the threshold range which is not lower than 40 times/minute and not higher than 60 times/minute.

Having determined that each of the respiration parameters (each of the parameters relevant to the ventilation volume, the parameters relevant to the airway pressure and the parameter relevant to the respiration rate) is included in the threshold range (YES in the step S3), the controller 2 executes processing of a step S4. On the other hand, having determined that at least one of the respiration parameters is outside the threshold range (NO in the step S3), the controller 2 executes processing of a step S5.

Incidentally, in the determination processing of the step S3, the controller 2 may determine whether at least one of the respiration parameters is included or not in the threshold range. For example, in the determination processing of the step S3, the controller 2 may only determine whether the respiration rate RR is included or not in the threshold range. In this case, the processing of the step S4 is executed when the respiration rate RR is included in the threshold range. On the other hand, the processing of the step S5 is executed when the respiration rate RR is outside the threshold range. In addition, the threshold range of each of the aforementioned respiration parameters is merely an example. In addition, the threshold range of each of the respiration parameters may be changed suitably in accordance with attributes (gender, age, etc.) or a health condition of the patient.

Next, when the determination result of the step S3 is YES, the controller 2 controls the light emitting device 20 so that the light emitting device 20 emits white light (step S4). Specifically, the controller 2 transmits, to the light emitting device driving circuit 9, a control signal for lighting each of the red light emitting element 33a, the green light emitting element 33b and the blue light emitting element 33c. Next, the light emitting device driving circuit 9 transmits a lighting signal (PWM signal) to each of the red light emitting element 33a, the green light emitting element 33b and the blue light emitting element 33c based on the control signal transmitted from the controller 2. Thus, the light emitting device 20 emits, to the outside, the white light which is combined light of the red light, the green light and the blue light.

On the other hand, when the determination result of the step S3 is NO, the controller 2 controls the light emitting device 20 so that the light emitting device 20 emits yellow light (step S5). Specifically, the controller 2 transmits, to the light emitting device driving circuit 9, a control signal for lighting the red light emitting element 33a and the green light emitting element 33b. Next, the light emitting device driving circuit 9 transmits a lighting signal to each of the red light emitting element 33a and the green light emitting element 33b based on the control signal transmitted from the controller 2. Thus, the light emitting device 20 emits the yellow light which is combined light of the red light and the green light toward the outside. Thus, the operations of the steps S1i to S5 are repeatedly executed.

According to the present embodiment, the visual aspect of the light emitting device 20 is changed in accordance with the result of the determination as to whether each of the respiration parameters of the patient is included or not in the threshold range. Further, the light emitting device 20 is removably attached to the measurement tube 140 of the respiration sensor 14. Therefore, by visually recognizing the visual aspect of the light emitting device 20 removably attached to the measurement tube 140, the medical worker who is in the middle of operating the ventilation device 30 can intuitively grasp abnormality of the respiratory condition of the patient even when the ventilation device or the respiration sensor without a light emitting unit (an indicator) is used.

Specifically, by visually recognizing the white light emitted from the light emitting device 20 even without confirming the physiological information displayed on the display unit 5 of the processing apparatus 1, the medical worker who is in the middle of operating the ventilation device 30 can intuitively grasp the situation that the respiratory condition of the patient is normal. On the other hand, by visually recognizing the yellow light emitted from the light emitting device 20 even without confirming the physiological information displayed on the display unit 5, the medical worker who is in the middle of operating the ventilation device 30 can intuitively grasp the situation that the respiratory condition of the patient is abnormal. Thus, the light emitting device 20 can visually present, to the medical worker, information indicating whether the respiratory condition of the patient is normal or abnormal.

In addition, according to the present embodiment, the light emitting elements 33 of the light emitting device 20 can emit red light, green light and blue light (i.e. lights of RGB which are three primary colors of light). Accordingly, the light emitting device 20 can emit light in various colors toward the outside.

Incidentally, in the description of the present embodiment, the light emitting device 20 is configured to emit light in different colors in accordance with the determination result of the step S3 (i.e. the respiratory condition of the patient). However, the present embodiment is not limited thereto. For example, the light emitting device 20 may be lighted/extinguished in accordance with the determination result of the step S3. Specifically, when the determination result of the step S3 is YES, the controller 2 may extinguish the light emitting device 20. On the other hand, when the determination result of the step S3 is NO, the controller 2 may light the light emitting device 20.

Further, blinking speed of the light emitting device 20 may change in accordance with the determination result of the step S3. Specifically, when the determination result of the step S3 is YES, the controller 2 may blink the light emitting device 20 at a first rate. On the other hand, when the determination result of the step S3 is NO, the controller 2 may blink the light emitting device 20 at a second rate which is different from the first rate.

Further, in the present embodiment, as shown in FIG. 2, the light emitting device 20 is removably attached to the measurement tube 140 configured as the columnar body. However, the present embodiment is not limited thereto. The light emitting device 20 may be removably attached to a portion (e.g. the air duct 35) of the ventilation device 30 configured as the columnar body. In addition, the light emitting device 20 may be removably attached to a cable configured as a columnar body or an arm of a bed.

(Specific Structure of Light Emitting Apparatus 20)

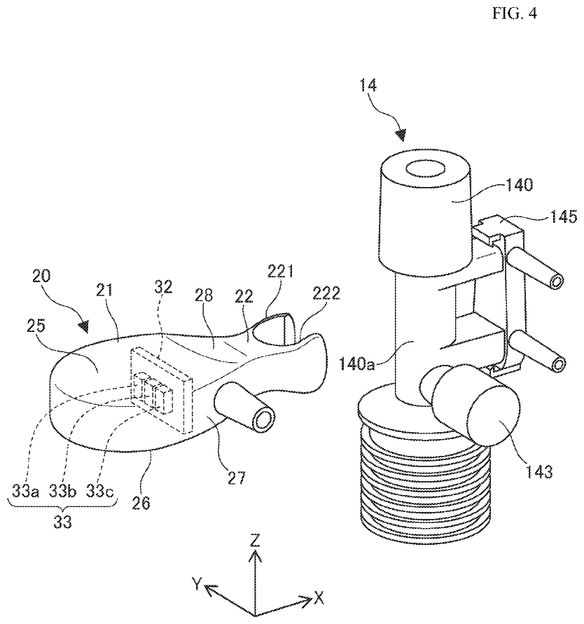

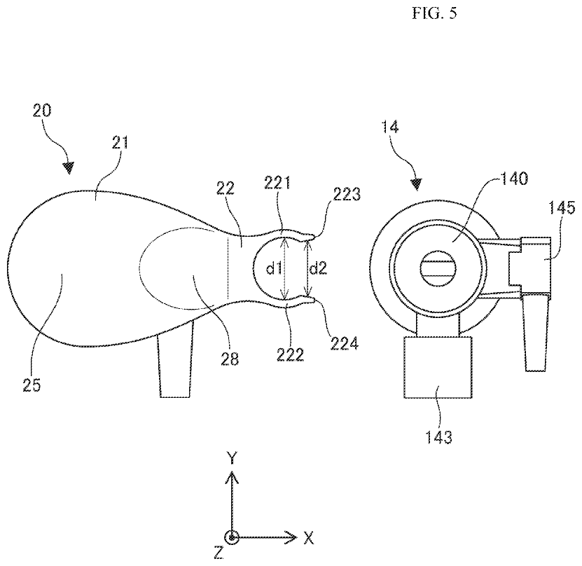

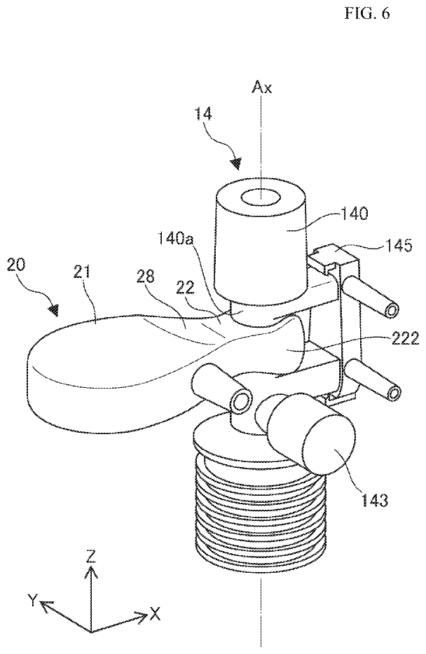

Next, the specific structure of the light emitting device 20 according to the present embodiment will be described below with reference to FIG. 4 to FIG. 6. FIG. 4 is a perspective view illustrating the light emitting device 20 and the measurement tube 140 of the respiration sensor 14. FIG. 5 is a top view illustrating the light emitting device 20 and the measurement tube 140. FIG. 6 is a perspective view illustrating the light emitting device 20 attached to the measurement tube 140.

As shown in FIG. 4, the light emitting device 20 is provided with the light emitting elements 33, a support substrate 32, a housing portion 21, and an attachment portion 22. The respiration sensor 14 has the measurement tube 140, the connection portion 145, a cap 143, and the inspiration side air tube with the expiration side air tube (not shown).

The light emitting elements 33 are configured by the red light emitting element 33a, the green light emitting element 33b, and the blue light emitting element 33c. The light emitting elements 33 are electrically connected to the processing apparatus 1 through an electric cable (not shown) which has been connected to the light emitting device 20. The support substrate 32 is configured to have the light emitting elements 33 mounted thereon. The housing portion 21 houses the light emitting elements 33 and the support substrate 32. The housing portion 21 is configured to allow at least a portion of light emitted from the light emitting elements 33 to pass through the housing portion 21.

The housing portion 21 has an upper face 25 (an example of a first face), a lower face 26 (an example of a second face), and a side face 27. The lower face 26 is opposed to the upper face 25 with the light emitting elements 33 interposed therebetween in a Z-axis direction. The side face 27 is positioned between the upper face 25 and the lower face 26. The housing portion 21 is, for example, formed out of a transparent resin. The upper face 25 and the lower face 26 of the housing portion 21 are embossed. Thus, light emitted from the light emitting elements 33 can pass through the upper face 25 and the lower face 26, and can be scattered on the upper face 25 and the lower face 26.

In addition, a recess portion 28 recessed along the shape of a finger of the medical worker is formed in the upper face 25. In addition, the support substrate 32 and the light emitting elements 33 are disposed inside the housing portion 21 so that the surface of the support substrate 32 on which the light emitting elements 33 is mounted is substantially perpendicular to the upper face 25 and the lower face 26. In this manner, light emitted from the light emitting elements 33 passes through the upper face 25 and the lower face 26. Accordingly, the medical worker can sufficiently visually recognize light emission of the light emitting device 20 even when the light emitting device 20 which has been turned upside down is attached to the measurement tube 140.

The attachment portion 22 which is connected to the housing portion 21 is configured to be attached to a small diameter portion 140a of the measurement tube 140. The attachment portion 22 may be formed out of one and the same resin as that of the housing portion 21. Particularly, the attachment portion 22 and the housing portion 21 may be formed integrally by resin molding.

In the case where the attachment portion 22 and the housing portion 21 form one piece construction, the attachment portion 22 and the housing portion 21 do not have to be disassembled when the light emitting device 20 is cleaned. Thus, maintenance performance of the light emitting device 20 can be improved, and manufacturing cost of the light emitting device 20 can be reduced.

The configuration of the attachment portion 22 will be described in detail with reference to FIG. 5. As shown in FIG. 5, the attachment portion 22 has a first elastic piece 221 (an example of a first piece), a first front end portion 223 connected to the first elastic piece 221, a second elastic piece 222 (an example of a second piece) opposed to the first elastic piece 221 with a gap inbetween in a Y-axis direction, and a second front end portion 224 connected to the second elastic piece 222.

In a state in which the attachment portion 22 has been attached to the small diameter portion 140a of the measurement tube 140, the small diameter portion 140a is pinched by the first elastic piece 221 and the second elastic piece 222. Particularly, when the attachment portion 22 is attached to the small diameter portion 140a, both the first elastic piece 221 and the second elastic piece 222 are elastically deformed so that the small diameter portion 140a can be pinched by the first elastic piece 221 and the second elastic piece 222. Thus, the light emitting device 20 can be removably attached to the measurement tube 140 due to the elastic deformation of the first elastic piece 221 and the second elastic piece 222. Incidentally, although both the first elastic piece 221 and the second elastic piece 222 are elastically deformed in the present embodiment, only one of the first elastic piece 221 and the second elastic piece 222 may be elastically deformed.

A distance d1 between the first elastic piece 221 and the second elastic piece 222 in the Y-axis direction may be slightly smaller than an outer diameter of the small diameter portion 140a. In addition, an interval d2 between the first front end portion 223 and the second front end portion 224 in the Y-axis direction increases gradually as the attachment portion 22 is away from the housing portion 21. In other words, the interval d2 gradually increases toward a +X direction. Thus, the medical worker can smoothly attach the light emitting device 20 to the measurement tube 140.

In addition, as shown in FIG. 6, the light emitting device 20 removably attached to the measurement tube 140 is attached to the measurement tube 140 rotatably around an axis Ax of the measurement tube 140. Thus, the medical worker can move the position of the housing portion 21 of the light emitting device 20 by rotating the light emitting device 20 around the axis Ax without detaching the light emitting device 20 from the measurement tube 140.

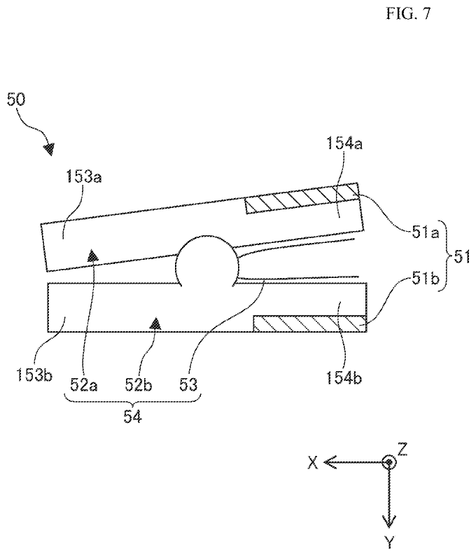

Next, a light emitting device 50 according to a modification of the present embodiment will be described below with reference to FIG. 7. FIG. 7 is a schematic view illustrating the light emitting device 50 according to the modification. As shown in FIG. 7, the light emitting device 50 having a configuration corresponding to a clothes pin is provided with a housing portion 51 and an attachment portion 54. The housing portion 51 has a first housing portion 51a and a second housing portion 51b. Each of the first housing portion 51a and the second housing portion 51b houses a light emitting element (not shown) configured to emit light, and is configured to allow at least a portion of the light emitted from the light emitting element to pass through the housing portion 51a, 51b. Thus, a visual aspect of the first housing portion 51a and the second housing portion 51b changes according to a respiratory condition of a patient.

The attachment portion 54 which is connected to the first housing portion 51a and the second housing portion 51b is configured to be attached to a columnar body such as the measurement tube 140. The attachment portion 54 has a first piece 52a, a second piece 52b opposed to the first piece 52a in a Y-axis direction, and a spring 53 (an example of an elastic body) disposed between the first piece 52a and the second piece 52b.

The first piece 52a which is connected to the first housing portion 51a has a clamp portion 153a which is attached to the not-shown columnar body, and an operation portion 154a to which external force can be given from a finger of a medical worker. In a similar manner or the same manner, the second piece 52b which is connected to the second housing portion 51b has a clamp portion 153b which is attached to the columnar body, and an operation portion 154b to which external force can be given from another finger of the medical worker. The first piece 52a is connected to the second piece 52b through a fulcrum 55.

The spring 53 is configured to give restoring force to the first piece 52a and the second piece 52b so that the first piece 52a and the second piece 52b can pinch the columnar body. Specifically, when an interval between the operation portion 154a and the operation portion 154b in the Y-axis direction is shortened by the external force given from the fingers of the medical worker, the spring 53 is deformed in the Y-axis direction. Thus, elastically restoring force of the spring 53 is given to at least one of the first piece 52a and the second piece 52b due to the elastic deformation of the spring 53. As a result, the light emitting device 50 can pinch the columnar body by the elastically restoring force of the spring 53, and is removably attached to the columnar body.

Although the embodiment of the present invention has been described above, the technical scope of the present invention should not be limitedly interpreted by the description of the present embodiment. The present embodiment is merely an example. It can be understood by those skilled in the art that various changes can be made on the embodiment within the scope of the invention stated in the scope of Claims. The technical scope of the present invention should be determined based on the scope of the invention stated in the scope of Claims and scopes of its equivalents.

In the description of the present embodiment, the visual aspect of the light emitting device 20 is changed in accordance with the determination result (the respiratory condition of the patient) of the step S3 shown in FIG. 3. However, the present embodiment is not limited thereto. In this respect, the light emitting device 20 may be configured to provide information indicating abnormality of the respiratory condition of the patient and/or operation guide information for guiding an operation timing of the ventilation device 30.

As an example of the operation guide information, the light emitting device 20 may provide the operation guide information for guiding the operation timing of the respiration bag 36 of the ventilation device 30. In this case, the medical worker can intuitively grasp the operation timing of the ventilation device 30 by visually recognizing the operation guide information. For example, assume that the light emitting device 20 blinks 50 times in one minute. In this case, the medical worker can carry out appropriate positive pressure ventilation on the patient by squeezing the respiration bag 36 in accordance with each lighting timing of the light emitting device 20.

In addition, when the respiratory condition of the patient is normal, the light emitting device 20 may blink white light at a predetermined blinking rate (e.g. 50 times/minute). On the other hand, when the respiratory condition of the patient is abnormal, the light emitting device 20 may blink yellow light at the predetermined blinking rate. In addition, the light emitting device 20 may be not electrically connected to the processing apparatus 1 when only the operation guide information is presented to the outside.

This application is based on Japanese Patent Application No. 2019-187882 filed on Oct. 11, 2019, the entire contents of which are incorporated herein by reference.

1. A physiological information processing system comprising:
a respiration sensor that is configured to detect a respiratory condition of a subject to whom a ventilation device is attached;
a light emitting device that is configured to emit light toward an outside; and
a physiological information processing apparatus that is connected to the respiration sensor and the light emitting device,
wherein the physiological information processing apparatus is configured to:
acquire a parameter relevant to respiration of the subject;
determine whether or not the parameter is included in a threshold range; and
change a visual aspect of the light emitting device in response to determining whether or not the parameter is included in the threshold range,
wherein the light emitting device is removably attached to a columnar body.

2. The physiological information processing system according to claim 1, wherein the columnar body is provided in the ventilation device or the respiration sensor.

3. The physiological information processing system according to claim 2, wherein the light emitting device comprises:
a light emitting element that is configured to emit light;
a housing portion that houses the light emitting element and that allows at least a portion of the light emitted from the light emitting element to pass through the housing portion; and
an attachment portion that is connected to the housing portion and is attached to the columnar body.

4. The physiological information processing system according to claim 3, wherein the attachment portion and the housing portion form one piece construction.

5. The physiological information processing system according to claim 3, wherein the light emitting element comprises:
a red light emitting element that emits red light,
a green light emitting element that emits green light, and
a blue light emitting element that emits blue light.

6. The physiological information processing system according to claim 3, wherein:
the attachment portion has a first piece, and a second piece that is opposed to the first piece;
the columnar body is pinched by the first piece and the second piece in a state where the attachment portion is attached to the columnar body; and
at least one of the first piece and the second piece is elastically deformed when the attachment portion is attached to the columnar body.

7. The physiological information processing system according to claim 6, wherein the attachment portion further comprises:
a first front end portion that is connected to the first piece; and
a second front end portion that is connected to the second piece and is opposed to the first front end portion, and
wherein an interval between the first front end portion and the second front end portion gradually increases as the attachment portion is away from the housing portion.

8. The physiological information processing system according to claim 3, wherein:
the housing portion comprises a first face, and a second face that is opposed to the first face via the light emitting element; and
the light emitted from the light emitting element passes through the first face and the second face.

9. The physiological information processing system according to claim 3, wherein the attachment portion comprises:
a first piece,
a second piece that is opposed to the first piece, and
an elastic body configured to apply a restoring force to the first piece and the second piece such that the first piece and the second piece pinch the columnar body.

10. The physiological information processing system according to claim 2, wherein the light emitting device is attached to the columnar body rotatably around an axis of the columnar body.

11. The physiological information processing system according to claim 1, wherein the parameter relevant to the respiration of the subject comprises at least one of:
a parameter relevant to a ventilation volume of the subject;
a parameter relevant to airway pressure of the subject; and
a parameter relevant to a respiration rate of the subject.

12. The physiological information processing system according to claim 1, wherein:
when the parameter is outside the threshold range, the physiological information processing apparatus controls the light emitting device such that the light emitting device emits light in a first light emission color; and
when the parameter is included in the threshold range, the physiological information processing apparatus controls the light emitting device such that the light emitting device emits light in a second light emission color different from the first light emission color.

13. A light emitting device that is removably attached to a columnar body, the light emitting device comprising:
a light emitting element that is configured to emit light;
a housing portion that houses the light emitting element and that allows at least a portion of the light emitted from the light emitting element to pass through the housing portion; and
an attachment portion that is connected to the housing portion and that is attached to the columnar body,
wherein the light emitting device is configured to provide information indicating abnormality of a respiratory condition of the subject or operation guide information for guiding an operation timing of the ventilation device.

14. A physiological information processing apparatus that is connected to a light emitting device and a respiration sensor,
wherein the light emitting device is configured to emit light toward an outside, and the respiration sensor is configured to detect a respiratory condition of a subject to whom a ventilation device is attached,
wherein the physiological information processing apparatus is configured to: acquire a parameter relevant to respiration of the subject;
determine whether or not the parameter is included in a threshold range; and
change a visual aspect of the light emitting device in response to determining whether or not the parameter is included in the threshold range,
wherein the light emitting device is removably attached to a columnar body.

15. A physiological information processing system comprising:
a light emitting device that is configured to emit light toward an outside; and
a physiological information processing apparatus that is connected to the light emitting device,
wherein the physiological information processing apparatus is configured to:
acquire a parameter relevant to respiration of a subject,
determine whether or not the parameter is included in a threshold range; and
change a visual aspect of the light emitting device in response to determining whether or not the parameter is included in the threshold range,
wherein the light emitting device is removably attached to a columnar body.
